(19) United States
(12) Patent Application Publication
(10) Pub. No.: US 2024/0122837 A1
(43) Pub. Date: Apr. 18, 2024

(54) PROCESS

(71) Applicant: GIVAUDAN SA, Vernier (CH)

(72) Inventors: Romain REYNAUD, Toulouse (FR); Bénédicte SENNELIER PORTET, Jonquerettes (FR); Mathieu TENON, Malemort du Comtat (FR)

(73) Assignee: GIVAUDAN SA, Vernier (CH)

(21) Appl. No.: 18/276,331

(22) PCT Filed: Mar. 17, 2022
(86) PCT No.: PCT/EP2022/057001

§ 371 Date: Aug. 8, 2023

(30) Foreign Application Priority Data
Mar. 19, 2021 (GB) 2103822.9
Mar. 16, 2022 (CH) CH00281/2022

(51) Int. Cl. A61K 8/67; A61K 8/365; A61K 8/92; A61K 8/9789; A61Q 19/08; C12N 1/16; C12P 15/00; C12P 23/00

(52) U.S. Cl. CPC A61K 8/671; A61K 8/365; A61K 8/922; A61K 8/9789; A61Q 19/08; C12N 1/16; C12P 15/00; C12P 23/00; A61K 2800/85

(57) ABSTRACT

The present invention relates to a process for preparing a composition comprising retinol, an oil and carnosic acid, where the carnosic acid reduces or prevents the oxidation of retinol and/or enhances the oxidative stability of retinol. The present invention also relates to compositions obtainable by the process and formulations containing said compositions, as well as to methods of reducing or preventing oxidation of retinol and/or enhancing the oxidative stability of retinol.

### FIELD OF THE INVENTION

The present invention relates to a process for preparing a composition comprising retinol, an oil and carnosic acid, where the carnosic acid reduces or prevents the oxidation of retinol and/or enhances the oxidative stability of retinol. The present invention also relates to compositions obtainable by the process and formulations containing said compositions, as well as to methods of reducing or preventing oxidation of retinol and/or enhancing the oxidative stability of retinol.

### BACKGROUND OF THE INVENTION

Retinol is one of the main anti-ageing actives used in cosmetics. Nevertheless, this molecule is not stable, and therefore requires antioxidant protection. Typically, this is through the use of a synthetic antioxidant: butylhydroxylanisol (BHA) and/or butylated hydroxytoluene (BHT) or other anti-oxidants such as tocopherol or tocopherol acetate. However, although the use of BHA and BHT is authorized in cosmetic regulation, there is a scientific debate on their toxicity and action as endocrine disruptors (https://www.febea.fr/fr/baseingredient/bht).

This has resulted in an increasing public demand for natural alternatives to artificial antioxidants that may be able to stablise and/or prevent the oxidation of retinol.

To this end, spices or plant juices or plant extracts or plant products may be used as antioxidants. One advantage of the use of such extracts or compounds is that they are perceived as natural ingredients when compared to antioxidants such as butylhydroxylanisol (BHA) and butylated hydroxytoluene (BHT). However, spices and plant juices or extracts or other products derived from spices/plants may contain components that may discolour or colour compositions/formulations they are incorporated into, which may prove undesirable to consumers.

Several different plant materials, such as fruit, vegetables, crops, herbs and spices have been investigated as potential sources of antioxidant compounds. Antioxidant compounds are typically present in plant material due to their importance in aiding the plants' growth and development.

However, the antioxidant efficacy of natural plant material varies greatly. In view of this, it is not a simple matter to predict which plant material or plant extracts would provide sufficient stabilising/antioxidant activity.

Additionally, in some cases, the amount of plant material or plant extract required to provide sufficient stabilising/antioxidant activity may prove uneconomical or be at a level which would be unacceptable due to regulatory and legislation constraints.

Retinol may not only be obtained through chemical synthesis, but also using biotechnology (e.g. by bioconversion or biosynthesis including, for example, a fermentation step). Retinol obtained using biotechnological methods including bioconversion and/or biosynthesis (e.g. by bioconversion or biosynthesis including, for example, a fermentation step in a culture medium) is typically called “bio-retinol”.

To this end, US 2022/017878 A1 describes a method of producing bio-retinol using a suitable microorganism. Such methods typically involve culturing a microorganism capable of providing retinol in a culture medium comprising an oil so that the retinol accumulates in the oil. The disclosure of US 2022/017878 A1 with respect to the production of bio-retinol is herewith incorporated by reference.

Oxidation of retinol may occur already during its synthesis, also during a bioconversion or biosynthesis. It is, therefore, necessary to stabilize the nascent bio-retinol already in the culture medium and/or oil.

The present invention seeks to address this problem by replacing synthetic antioxidants traditionally used to stabilize and/or prevent oxidation in retinol by a natural ingredient, such as a rosemary extract highly titrated in carnosic acid.

Carnosic acid is a labdane-type diterpene present in plant species of the Lamiaceae family, such as rosemary (Rosmarinus officinalis) and common salvia (Salvia officinalis; Hossain et al., 2010; Birtie et al., 2015).

While the potential antioxidative activity of carnosic acid is well documented, its exact mechanism of action has not been studied extensively. In particular, little is known of the interactions of carnosic acid with distinct reactive oxygen species (ROS) or lipid radicals.

Moreover, in most studies, in vitro oxidation was generated by prolonged and artificial heating treatments, so that it is difficult to extrapolate the results to the in vivo situation in plants. Surprisingly, the role of carnosic acid in plant leaves has received little attention, and the biological role of this compound in plants is not firmly established.

Carnosic acid is present at very high concentrations, up to several percent of dry weight, in leaves of the Mediterranean half-shrub rosemary (Munné-Bosch and Alegre, 2001; del Baño et al., 2003; Luis and Johnson, 2005). Carnosic acid biosynthesis and accumulation take place exclusively in young rosemary leaves at the branch apices, with the diterpene molecule being partially consumed during leaf development and aging (Hidalgo et al., 1998; Bruckner et al., 2014; Božić et al., 2015). Beside carnosic acid, less abundant phenolic diterpenes can be measured in rosemary leaves, including carnosol, the major oxidation product of carnosic acid. The antioxidative activity of the latter compound, produced spontaneously from carnosic acid by nonenzymatic reaction, has been seldom investigated (Aruoma et al., 1992; Zeng et al., 2001). Diterpene levels in field-grown rosemary plants displayed seasonal changes, with a tendency for carnosic acid losses in response to environmental stress conditions (Luis and Johnson, 2005). In particular, carnosic acid concentrations in rosemary leaves under natural conditions were found to decrease at high temperatures and low precipitation rates in summer with concomitant increases in oxidized derivatives, suggesting that cellular oxidative stress is accompanied by the consumption of carnosic acid (Munné-Bosch et al., 1999; Munné-Bosch and Alegre, 2003). Both carnosic acid and carnosol accumulate in photosynthetic green tissues only (leaves, sepals, and petals) and have be localized in the chloroplasts (Munné-Bosch and Alegre, 2001), although the synthesis of carnosic acid also has been reported in glandular trichomes (Bruckner et al., 2014).

### DISCLOSURE OF THE INVENTION

### Process for Preparing the Composition of the Invention

The present inventors have surprisingly and unexpectedly found that carnosic acid can reduce or prevent the oxidation of retinol and/or enhance the oxidative stability of retinol even during its synthesis, in particular using biotechnology.

Therefore, in a first aspect, present invention provides a process for preparing a composition comprising retinol, an oil and carnosic acid. The process comprises the step of culturing a microorganism capable of providing retinol in a culture medium comprising an oil so that retinol accumulates in the oil, wherein carnosic acid is added to the culture medium.

This process is hereinafter referred to as the process of the invention.

Specific and preferred embodiments described for one aspect of the invention also apply to the other aspects of the invention.

When we use the term “consisting essentially of” or “consists essentially of” we mean that the composition or extract or juice being described must contain the listed ingredient(s) and may also contain small (for example up to 2% by weight, or up to 1% or up to 0.1% or 0.01% by weight) of other ingredients provided that any additional ingredients do not affect the essential properties of the composition, extract or juice. When we use the term “consisting of” we mean that the composition being described must contain the listed ingredient(s) only. These terms can be applied in an analogous manner to processes, methods and uses.

It is also intended that the terms “comprise” or “comprises” or “comprising” may be replaced with “consist” or “consisting” or “consisting” throughout the application.

The term “about” as used herein, e.g. when referring to a measurable value (such as an amount of weight of a particular component in the composition or reaction mixture), refers to variations of ±20%, ±10%, ±5%, ±1%, ±0.5%, or particularly, ±0.1%, of the specified amount.

Retinol has the formula as shown in Formula 1:

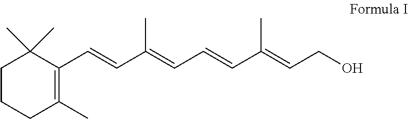 [SMILES: CC1=C(/C=C/C(C)=C/C=C/C(C)=C/CO)C(C)(C)CCC1]

Retinol, as prepared in the process of the invention, may exist in the trans and/or cis isomeric form. As used herein, the term “retinol” is intended to encompass both trans and/or cis forms of retinol.

In the process and compositions of the invention, it may be preferred that at least 90% of the retinol is trans-retinol, such as at least 95% or at least 99% or 99.5% of the retinol is trans-retinol. For example, in the process and compositions of the invention, from about 90% to about 99.5% of the retinol is trans-retinol.

The term “retinol”, as used herein, is also intended to cover derivatives of retinol, such as ester derivatives. For example, retinol derivatives may include, but are not limited to: retinyl palmitate, retinyl acetate, retinyl propionate, retinyl linoleate, retinyl oleate, retinyl rice branate, retinyl soyate, retinyl tallate, retinal and mixtures thereof.

In the process of the invention, the retinol is obtained by biotechnology (e.g. by bioconversion or biosynthesis including, for example, a fermentation step). Alternatively or in addition, it could also be obtained through chemical synthesis.

Microorganisms capable of providing retinol are generally known in the art. For example, such microorganisms are described in US 2022/017878 A1.

As used herein, the term “fermentation” refers to the bulk growth of microorganisms in a growth medium with the goal of producing a natural product (such as, for example, retinol).

As used herein, the term “bioconversion” means an enzyme-catalysed process where an intermediate or precursor is converted into a target end product (such as, for example, retinol)

As used herein, the term “biosynthesis” means a multi-step, enzyme-catalysed process where substrates (e.g. carbon substrates) are converted into a more complex product (such as, for example, retinol) in living organisms (such as, for example, microorganisms).

Throughout this disclosure, the terms “culturing” and “fermenting” are used interchangeably.

In the process of the invention, the retinol produced is bio-retinol.

The oil used in the process of the invention may be any oil, but typically is an oil obtained or obtainable from plants and/or animals. For example, the oil may comprise one or more plant oils or vegetable oils selected from one or more of coconut oil, palm oil, cottonseed oil, wheat germ oil, soybean oil, sesame oil, olive oil, corn oil, canola oil, sunflower oil (such as, for example, mid-oleic sunflower oil and/or high oleic sunflower oil and/or organic sunflower oil and/or sunflower seed oil), safflower oil, peanut oil, flaxseed oil, grape seed oil, and rapeseed oil, botanical oil and mixtures thereof. Preferably, the oil is sunflower oil.

As will be appreciated by the person skilled in the art, as used herein, the term “obtainable from” means that the extract may be obtained from a plant or may be isolated from the plant, or may be obtained from an alternative source, for example by chemical synthesis or enzymatic production. Whereas the term “obtained” as used herein, means that the extract is directly derived from the plant source.

In the process of the invention, the carnosic acid may be provided in the form of an extract obtained or obtainable from at least one plant selected from the Lamiaceae family, such as rosemary and/or sage.

The extract obtained or obtainable from at least one plant selected from the Lamiaceae family, such as rosemary and/or sage may be in the form of a liquid or a solid. Typically, the extract may be in the form of a solid, i.e. a powder.

All references herein to an extract obtained from or obtainable from a plant from the Lamiaceae family (such as rosemary and/or sage) will typically refer to extracts obtained from or obtainable from the leaves of the plant from the Lamiaceae family. The leaves may have been dried and/or ground before being extracted. The Lamiaceae extract may be deodorised and/or decolourised. For example, the Lamiaceae extract may have had all of the volatile oil compounds removed.

In some aspects, the at least one plant selected from the Lamiaceae family will have been extracted from the respective plants using water only. For example, the at least one plant selected from the Lamiaceae family may have been extracted using water only. This extract may be referred to as the water extract.

In certain aspects, the water used to provide the extract may be acidic. For example, the pH of the water used to provide the extract may be from about 1 to about 6, or from about 2 to about 4, such as pH 3. This extract may be referred to as the acidic water extract.

In other aspects, the at least one plant selected from the Lamiaceae family may have been extracted from the respective plants using alcohol, such as ethanol. For example, the at least one plant selected from the Lamiaceae family may have been extracted using alcohol only, such as ethanol only. This extract may be referred to as the alcohol extract, such as the ethanol extract.

In other aspects, the at least one plant selected from the Lamiaceae family may have been extracted from the respective plants using a mixture of alcohol and water, such as ethanol and water. For example, the at least one plant selected from the Lamiaceae family may have been extracted using a mixture of alcohol and water, such as ethanol and water. This extract may be referred to as the hydro-alcoholic extract, such as the hydro-ethanolic extract.

In other aspects, the at least one plant selected from the Lamiaceae family may have been extracted from the respective plants using an organic solvent that is not an alcohol, such as acetone. For example, the at least one plant selected from the Lamiaceae family may have been extracted using an organic solvent, such as acetone. This extract may be referred to as the organic extract or the acetone extract.

For the avoidance of doubt, preferences, options, particular features and the like indicated for a given aspect, feature or parameter of the invention should, unless the context indicates otherwise, be regarded as having been disclosed in combination with any and all other preferences, options, particular features and the like as indicated for the same or other aspects, features and parameters of the invention.

The extract obtained from or obtainable from a plant of the Lamiaceae family comprises carnosic acid. For example, the extract obtained from or obtainable from a plant of the Lamiaceae family may comprise carnosic acid in an amount of at least 1% by weight of the extract. For example, the extract obtained from or obtainable from a plant of the Lamiaceae family may contain carnosic acid in an amount of from about 1% to about 95% by weight, such as from about 2.5% to about 90% by weight, from about 5% to about 85% by weight, from about 10% to about 80% by weight, from about 15% to about 60% by weight, from about 20% to about 50% by weight, from about 25% to about 40% by weight, such as from about 1% to about 30% by weight, from about 5% to about 20% by weight, from about 10% to about 15% by weight or from about 1% to about 5% by weight, e.g. from about 15% to about 30% by weight of the extract or from about 40% to about 65% by weight of the extract. For example, the carnosic content in the extract obtained or obtainable from rosemary may be at least about 20% by weight of the rosemary extract.

The extract obtained from or obtainable from a plant of the Lamiaceae family may also comprise carnosol and 12-O-methylcarnosic acid. For example, the extract obtained from or obtainable from a plant of the Lamiaceae family may comprise or contain from about 2% to about 10% carnosol and from about 2% to about 10% by weight 12-O-methylcarnosic acid.

As will be appreciated by the person skilled in the art, the amount of extract required will depend on the concentration of carnosic acid present in the extract and the amount of carnosic acid intended to be used in the process and composition of the invention. For example, where the extract comprises 50% carnosic acid by weight of the extract, 1% of the extract would need to be used in order to obtain a concentration of 0.5% in the medium.

Unless otherwise stated herein, the weight percentages listed are based on the total weight of the extract, for example the total weight of the dry extract.

The carnosic acid may be added to the culture medium at any suitable point in time.

In an embodiment, the carnosic acid is added to the culture medium at the start of and/or during the culturing.

The carnosic acid may be added to the culture medium separately or in combination with other components, for example together with an oil, in particular with (part of) the oil (e.g. sunflower oil) used in the culturing medium.

The carnosic acid may be diluted in an oil or other suitable diluent or solvent (or mixtures thereof) prior to its addition to the culture medium. Such oil or diluent or solvent should be compatible with the other components used in the culturing, in particular the microorganism, retinol and retinol precursors and derivatives, as well as the culture medium and oil.

For example, the carnosic acid may be diluted in the same oil as provided in the culture medium. In this case, part of or all the oil may be provided together with the carnosic acid.

The carnosic acid is advantageously added in an amount that is not toxic to the microorganism.

The potential toxicity of carnosic acid to the microorganism can be determined by testing the effect of different concentrations of carnosic acid on the microorganism during the culturing phase. Such testing is routine work for the skilled person.

The oil may be at a temperature of from about 40° C. to about 60° C., such as from about 45° C. to about 55° C.

The mixture of oil, retinol and carnosic acid may then be agitated or stirred (for example as part of a microbial culture in a bioreactor) until the retinol and carnosic acid have dissolved and accumulate in the oil.

On completion of the fermentation step, the mixture may then be filtered (to remove, for example, the biomass and other particulate matter) and decanted after phase separation in order to recover the retinol containing oil.

The process for preparing a composition of the invention includes adding the carnosic acid (and optionally other additional anti-oxidants) during the process of forming the bio-retinol (such as, for example, during a fermentation process).

For example, the carnosic acid (such as, for example in the form of an extract obtained or obtainable from at least one plant selected from the Lamiaceae family) may be added during the culturing of a microorganism that is capable of providing retinol (such as, for example, during a fermentation process).

Thus, the present invention further provides a process for the preparation of a composition of the invention, wherein the process comprises the following steps in any order:
(i) Culturing/fermenting a microorganism capable of providing retinol in a culture medium comprising an oil so that retinol accumulates in the oil;
(ii) Adding carnosic acid (such as, for example, in the form of an extract obtained or obtainable from at least one plant selected from the Lamiaceae family) to the culture medium (such as, for example at the start of and/or during the culturing/fermenting step (i)); and optionally
(iii) Isolating retinol (such as bio-retinol) and/or carnosic acid and/or oil.

The process may optionally include adding an additional anti-oxidant to the composition. This may be added during any one of steps (i) to (iii).

Preferably, no synthetic anti-oxidant is added during the process of the invention.

The nature of the microorganism is not limited, as long as it is a microorganism capable of producing retinol; but specifically, it may be Yarrowia lipolytica or Saccharomyces cerevisiae. The microorganism may be genetically modified to optimize the production of retinol. Examples of suitable microorganisms are described in US 2022/017878 A1, the disclosure of which in this context is herewith incorporated by reference.

It is known in the art that retinol can be produced from a microbial culture (for example, in a bioreactor (also termed a fermenter)), which method comprises culturing a microbial cell producing retinol for recovery from a culture medium, the bioreactor containing an aqueous phase and an extraction phase. The microbial cell is grown in an aqueous phase in a bioreactor, and may be cultured in batch culture, continuous culture, or semi-continuous culture. The aqueous phase generally comprises an appropriate cell culture medium, and may further comprise precursor molecules for production of the secondary metabolite. In some embodiments, carbon substrates are fed to the culture for production of the target retinol product.

The culture/fermentation process can be easily adjusted and used by a person skilled in the art according to the selected microorganism.

In the process of the invention, step (i) may be conducted in a culture medium in a bioreactor.

The culture medium and other culture conditions used for culturing/fermenting the microorganisms may be any medium without particular limitation as long as it is a medium used for cultivation of ordinary microorganisms. In a conventional medium containing amino acids and/or vitamins, it can be cultured while controlling temperature, pH, agitation conditions etc. under aerobic conditions.

In the culturing/fermentation step, the pH can be adjusted using a basic compound (e.g. sodium hydroxide, potassium hydroxide or ammonia) or an acidic compound (e.g. phosphoric acid or sulfuric acid) to provide a pH of 5.5 to 7.5, 5.5 to 7.0, or 6.0 to 7.5. For example, the pH may be 6.9.

In order to maintain the aerobic state, oxygen or oxygen-containing gas may be injected into the culture, or nitrogen, hydrogen or carbon dioxide gas may be injected without the injection of gas to maintain the anaerobic and microaerobic state.

The temperature may be maintained at from about 20° C. to about 45° C. or from about 25° C. to about 40° C., specifically from about 27° C. to about 31° C., more specifically about 30.2° C.

The operating rotational speed (rpm) of the incubator/bioreactor in the culturing/fermentation step may be from about 50 to about 300 rpm, such as from about 50 to about 250 rpm, or from about 100 to about 300 rpm, such as from about 200 to 300 rpm.

The term “medium” or “biomedium” means a culture medium for culturing the microorganism.

The bioreactor, in which the microorganisms producing retinol are grown, contains a culture medium, which has an aqueous phase that includes sugars and carbohydrates as carbon sources (e.g. glucose, sucrose, lactose, fructose, maltose, molasse, starch and cellulose), and an extraction phase containing fats (such as, but not limited to oils, e.g. coconut oil, palm oil, cottonseed oil, wheat germ oil, soybean oil, sesame oil, olive oil, corn oil, canola oil, sunflower oil (such as, for example, mid-oleic sunflower oil and/or high oleic sunflower oil and/or organic sunflower oil and/or sunflower seed oil), safflower oil, peanut oil, flaxseed oil, grape seed oil, and rapeseed oil, botanical oil and/or mixtures thereof, preferably sunflower oil), fatty acids (e.g. palmitic acid, stearic acid and linoleic acid), alcohols (e.g. glycerol and ethanol), organic acids (e.g. acetic acid) and mixtures thereof.

Nitrogen sources include nitrogen-containing organic compounds (e.g. peptone, yeast extract, broth, malt extract, corn steep liquor, soybean meal and urea), inorganic compounds (e.g. ammonium sulfate, ammonium chloride, ammonium phosphate, ammonium carbonate and ammonium nitrate) and mixtures thereof.

Potassium dihydrogen phosphate, dipotassium hydrogen phosphate, sodium-containing salts of the aforementioned and mixtures thereof may be used as a phosphorus source.

The culture medium may also contain essential growth-promoting substances, such as other metal salts (e.g. magnesium sulfate or iron sulfate), amino acids and vitamins.

The culture medium used for culturing/fermenting the microorganisms producing retinol may contain one or more nutrients selected from the group consisting of yeast extract, peptone, soy bean, and glucose.

The yeast extract may be included in an appropriate amount in the medium. For example, in an amount of from about 1 to about 4 parts by weight, such as from about 1.5 to about 4 parts by weight, or from about 2 to about 4 parts by weight, based on 100 parts by weight of the total culture medium.

The peptone may be included in an appropriate amount in the medium. For example, from about 0.5 to about 4 parts by weight, such as from about 1 to about 4 parts by weight, or from about 1.5 to about 4 parts by weight based on 100 parts by weight of the total culture medium.

The soybean may be included in an appropriate amount in the medium. For example, from about 0.5 to about 4 parts by weight, such as from about 1 to about 4 parts by weight, or from about 1.5 to about 4 parts by weight based on 100 parts by weight of the total culture medium.

The glucose may be included in an appropriate amount in the medium. For example, from about 1 to about 3 parts by weight, such as from about 1 to about 2.5 parts by weight, or from about 1.5 to about 3 parts by weight based on 100 parts by weight of the total culture medium.

The retinol produced is secreted into the culture medium.

As noted above, the bioreactor contains an aqueous phase and an extraction phase. The extraction may comprise an oil, such as sunflower oil. The extraction phase oil may be present in the bioreactor at the start of the culturing/fermentation or may be added to the bioreactor at an appropriate point, for example after at least about 1 hour of culturing/fermenting, or at least about 2 hours of culturing/fermenting or at least about 5 hours of culturing/fermenting.

The oil in the extraction phase, may comprise carnosic acid (i.e. in the form of an extract obtained or obtainable from at least one plant selected from the Lamiaceae family), or carnosic acid may be added to the oil at an appropriate point.

Thus, the present invention provides a process for providing a composition of the invention comprising:
(i) Culturing/fermenting a microorganism that is capable of providing retinol (such as, for example, Yarrowia lipolytica or Saccharomyces cerevisiae) in a culture medium comprising an oil so that retinol accumulates in the oil (optionally comprising carnosic acid (e.g. in the form of an extract obtained or obtainable from at least one plant selected from the Lamiaceae family));
(ii) Adding carnosic acid (i.e. in the form of an extract obtained or obtainable from at least one plant selected from the Lamiaceae family) to the medium if not already present; and optionally
(iii) Isolating retinol (bio-retinol) and/or carnosic acid and/or oil.

The oil in the extraction phase may comprise one or more plant oils or vegetable oils selected from one or more of coconut oil, palm oil, cottonseed oil, wheat germ oil, soybean oil, sesame oil, olive oil, corn oil, canola oil, sunflower oil (such as, for example, mid-oleic sunflower oil and/or high oleic sunflower oil and/or organic sunflower oil and/or sunflower seed oil), safflower oil, peanut oil, flaxseed oil, grape seed oil, rapeseed oil, and mixtures thereof. Preferably, the oil is sunflower oil.

If an additional anti-oxidant is required, for example BHT, BHA and/or tocopherols, these may be added at any step. For example, they may be added at the same time as carnosic acid is added.

The step of isolating retinol (such as, for example bio-retinol) and/or carnosic acid and/or oil from the process may be achieved by any means known in the art.

The step of recovering the retinol may additionally include a separation process and/or a purification step as required.

Thus, the present invention may provide a composition obtainable or obtained from the process of the invention comprising retinol (such as bio-retinol) preferably in the form of a liquid, such as an oily liquid (such as, for example bio-retinol in sunflower oil) and carnosic acid (such as at least 0.5% carnosic acid by weight of the composition).

The composition obtainable or obtained from the process of the invention is preferably a cosmetic composition.

The process of the invention may further comprise the step of formulating the composition comprising the retinol, oil and carnosic acid into a formulation or product.

Preferably, the formulation/product is a cosmetic formulation/product, and more preferably an anti-aging formulation/product.

### Compositions

The present inventors have surprisingly and unexpectedly found that a composition comprising retinol, an oil and carnosic acid can reduce or prevent the oxidation of retinol and/or enhance the oxidative stability of retinol.

Therefore, the present invention provides a composition comprising retinol, an oil and carnosic acid (such as at least 0.5% carnosic acid by weight of the composition).

Such compositions are hereinafter referred to as the “composition of the invention”.

Typically, the composition of the invention may be in the form of a liquid, such as an oily liquid.

The compositions of the present invention may comprise retinol and carnosic acid (such as at least 0.5% carnosic acid by weight of the composition) in the form of a liquid, such as an oily liquid.

In the composition of the invention, the amount of carnosic acid may be from about 0.1% to about 10% by weight of the composition, such as from about 0.5% to about 7% or from about 2% to about 5% by weight of the composition.

Alternatively, the composition of the invention may consist or consist essentially of retinol, an oil and carnosic acid.

Optionally, the composition of the invention may comprise, consist or consist essentially of the retinol, oil and carnosic acid combination(s) defined above and a carrier.

Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

The term “carrier” as used herein, may refer to a natural product or a product originating from nature that has been transformed or modified so that it is distinct from the natural product from which it originated.

Preferably, the retinol is in the form of bio-retinol as defined above.

As defined above, the carnosic acid may be provided in the form of an extract obtained or obtainable from at least one plant selected from the Lamiaceae family, such as rosemary and/or sage.

Suitable oils to be used in the composition of the invention are also defined above.

The retinol present in the composition of the invention may be present in an amount of from about 20% to about 80% by weight of the composition, such as from about 30% to about 70% by weight of the composition, preferably from about 40% to about 60% by weight of the composition or from about 40% to about 50% by weight of the composition.

The oil present in the composition of the invention may be present in an amount of from about 30% to about 70% by weight of the composition, such as from about 35% to about 60% by weight of the composition.

The compositions of the invention may further comprise an additional anti-oxidant. For example, the compositions of the invention may comprise BHT and/or BHA and/or tocopherols.

Thus, the present invention may also provide a composition comprising retinol (preferably bio-retinol), an oil (preferably sunflower oil), carnosic acid (preferably in the form of an extract obtained or obtainable from a plant of the Lamiaceae family), and further BHT and/or BHA and/or tocopherols. Preferably, the composition of the invention does not comprise any synthetic anti-oxidants.

If present, the additional anti-oxidant may be present in an amount from about 1% to about 5% by weight of the composition, such as from about 2% to about 4% by weight of the composition.

For example, the composition of the invention may comprise:
from about 40% to about 60% retinol (in particular, bio-retinol, optionally wherein from about 90% to about 99% of the retinol is trans-retinol);
from about 0.5% to about 7% carnosic acid (optionally wherein the carnosic acid is in the form of an extract obtained or obtainable from a plant of the Lamiaceae family);
from about 30% to about 70% of an oil (optionally where in the oil is sunflower oil); and optionally
from about 1% to about 5% of an additional anti-oxidant (optionally wherein the additional anti-oxidant is BHT, BHA, tocopherols or mixtures thereof).

In the compositions of the invention, it may be preferred that the compositions do not comprise certain components. For example, it may be preferred that the compositions do not comprise retinoic acid as this is an indication that the retinol present in the composition is not oxidatively stable.

Therefore, the present invention may also provide compositions as previously defined wherein the composition comprises less than 5% retinoic acid by weight of the composition, such as less than about 1% retinoic acid by weight of the composition, or less than about 0.5% retinoic acid by weight of the composition. Preferably, the compositions of the invention comprise no retinoic acid.

In the compositions of the invention, it may be preferred that the compositions do not comprise certain components, such as, for example, retinol isomers (such as, for example, 9-cis-retinol and/or 13-cis-retinol). For example, if retinol isomers are present, it may be preferred that the compositions comprise less than or equal to 5% of these retinol isomers.

As noted above, it has been surprisingly and unexpectedly found by the present inventors that in the compositions of the invention, comprising retinol, an oil and carnosic acid, for instance bio-retinol (in the form of a liquid, such as an oily liquid) and carnosic acid, the oxidation of retinol is reduced or prevented and/or the oxidative stability of retinol is enhanced.

In an embodiment, the composition comprises retinol, an oil and carnosic acid, wherein the carnosic acid is in the form of a hydro-ethanolic rosemary and/or sage leaf extract as previously defined above. Alternatively, it would also be possible to use other extraction solvents or solvent mixtures commonly used for extracting rosemary and/or sage, for instance acetone or acetone mixtures.

Thus, the present invention provides a composition (preferably an anti-oxidant composition) as defined previously, where the composition reduces or prevents the oxidation of retinol and/or enhances the oxidative stability of retinol.

As used herein, the term “reducing or preventing oxidation of retinol” is intended to mean that less than about 10% of the retinol present in the composition is oxidised to retinal and/or retinoic acid over a time period of at least 1 month, or at least 3 months or at least 6 months. For example, less than about 5% of the retinol present in the composition is oxidised to retinal and/or retinoic acid or less than 1% or less than 0.5% of the retinol present in the composition is oxidised to retinal and/or retinoic acid over a time period of at least 1 month, or at least 3 months or at least 6 months.

As used herein, the term “enhances the oxidative stability of retinol” is intended to mean that at least 90% of the retinol present in the composition is still in the form of retinol over a time period of at least 1 month, or at least 3 months or at least 6 months. For example, more than 95% of the retinol present in the composition is still in the form of retinol or more than 99% or 99.5% of the retinol present in the composition is still in the form of retinol over a time period of at least 1 month, or at least 3 months or at least 6 months.

### Formulations and Products

The composition of the invention may be incorporated into a formulation/product, such as a nutraceutical, pharmaceutical, veterinary, oenological or cosmetic formulation/product.

Thus, the present invention provides a formulation comprising a composition of the invention. For example, the present invention may provide a cosmetic composition comprising the composition of the invention.

The present invention also provides a product comprising a composition of the invention. For example, the present invention may provide a cosmetic product comprising the composition of the invention.

A “cosmetic product” is intended to mean any substance or mixture intended to be placed in contact with the external parts of the human body (epi-dermis, hair system, nails, lips and external genital organs) or with the teeth and the mucous membranes of the oral cavity with a view exclusively or mainly to cleaning them, perfuming them, changing their appearance, protecting them, keeping them in good condition, correcting body odours and/or combinations thereof.

A “substance” is intended to mean a chemical element and its compounds in the natural state or obtained by any manufacturing process, including any additive necessary to preserve its stability and any impurity deriving from the process used but excluding any solvent which may be separated without affecting the stability of the substance or changing its composition.

A “mixture” is intended to mean a mixture or solution composed of two or more substances.

The present invention also provides the use of a composition of the invention in a nutraceutical, pharmaceutical, veterinary, oenological or cosmetic formulation/product.

Such formulations or products are hereinafter referred to as the “formulations or products of the invention”.

The nutraceutical, pharmaceutical, veterinary, oenological or cosmetic formulation/product may optionally further comprise pharmaceutically/veterinary/cosmetic (including cosmetic active) ingredients, such as excipients, carriers and mixtures thereof as appropriate.

“Cosmetic” or “Cosmetic Active Ingredients” means any and all natural, naturally occurring, nature identical, synthetic synthetically produced, biosynthetically produced, sustainable, renewable and/or biodegradable compounds, ingredients, intermediates, molecules, substances, raw materials or products individually or as part of a mixture of compounds, ingredients, intermediates, molecules, substances, raw materials or products, blends, compositions, formulations (including but not limited to skin moisturizers, creams, balms, serums, oils, eye, facial makeup, wash off hair products, leave-on hair products, hair colorants (including but not limited to natural hair colorants) and/or combinations thereof), finished products and related technologies including, but not limited to components, incorporated, for example, into a cosmetic formulation (such as but not limited to natural colorants, preservatives, emulsifiers, anti-oxidants and the like which do not, for example, have an activity on the skin, hair, scalp and the like but play a role in the formulation of the finished product), delivery systems, marketing aids (such as, for example, coloured unispheres applied to translucent formulations) and methods of making anything related thereto useful in/used for/intended for:
application by rubbing, pouring, sprinkling, spraying or otherwise directly on or to a human or animal body and/or by placing in contact with the various external and/or on surface parts of a human or animal body (including but not limited to the skin, hair, body hair, the hair system, scalp, nails, lips, external genitalia, teeth, oral and/or nasal mucosa and the like); and/or application indirectly on or to a human or animal body such as for example, application as part of a textile or application to a textile as part of a delivery device (such as a capsule) or a delivery system (such as blends or formulations) applied to a textile; and/or
cleansing, caring, cooling, beautifying, conditioning, treating, soothing, texturizing, promoting attractiveness, protecting, maintaining, improving, enhancing, altering and/or changing an external part and/or surface of a human or animal body (such as, but not limited to the scalp) or the aesthetic appearance of a human or animal body; and/or with a view to mainly cleaning or perfuming, or protecting or maintaining in good condition or combating body odour or changing the appearance of or correcting or repairing a state of imbalance in skin, oral mucosa, scalp or hair by providing a calming, healing, repairing, or revitalization, hydration of the skin or in order to provide relief to, lubricate, moisten, tone, heal, sterilize, relieve, correct and/or remedy states of dryness, irritation, injury or fatigue, and/or with a view to correcting pigmentation disorders or providing a non-pharmaceutical prevention and/or treatment of dandruff, acne, irritation and/or inflammation and the like and/or rebalancing the bacterial flora (such as, for example, the microbiome) on the surface of the skin (such as, for example, by promoting the level of beneficial bacterial flora on the skin surface) and/or for the purposes of keeping a human or animal body in good condition for health and/or wellbeing purposes and/or for improving the appearance of a human or animal body by, for example, improving the appearance of a product applied to a human or body; and/or
providing a cosmetic and/or dermatological function and/or benefit with a biological activity benefit (but without affecting a body's structure or function. For the avoidance of doubt, a Cosmetic or Cosmetic Active Ingredient or any part thereof may also qualify as a Functional Ingredient and/or Nutraceutical.

“Functional Ingredient” means a food ingredient or part of a food that provides medicinal or health benefits, including any of the following: a carotenoid, dietary fiber, fatty acid, saponin, antioxidant, flavonoid, isothiocyanate, phenol, polyphenol (such as resveratrol), plant sterol or stanol (phytosterols and phytostanols), a polyol, a prebiotic, a phytoestrogen, soy protein, sulfides/thiol, a vitamin, glucosamine, preservatives, hydration agents, edible gelling ingredients, edible gel mixes and gel compositions, long chain primary aliphatic saturated alcohols, colour agents, texturizing agents, emulsifiers and combinations thereof.

“Nutraceutical” means any and all natural, naturally occurring, sustainable, synthetically-produced and biosynthetically-produced compounds, mixtures of compounds, Functional Ingredients, molecules, compositions, raw materials, and intermediates (including components and delivery devices (such as capsules) related thereto, delivery systems thereof (such as blends or formulations) and methods of making the foregoing) that are associated with health and/or cosmetic benefits, as well as improving or maintaining the appearance of the human body. For the avoidance of doubt, Nutraceuticals includes compounds that can be used as supplements to food or beverage, whether a solid formulation, capsule, tablet, liquid formulation, solution or suspension.

Alternatively, the nutraceutical, pharmaceutical, veterinary, oenological or cosmetic formulation/product may consist or consist essentially of the composition of the invention.

The cosmetic formulation/product may be an anti-aging formulation.

As used herein, references to pharmaceutically, veterinary or cosmetically acceptable excipients may refer to pharmaceutically, veterinary or cosmetically acceptable adjuvants, diluents and/or carriers as known to those skilled in the art.

By “pharmaceutically/veterinary/cosmetically acceptable” we mean that the additional components of the composition are generally safe, non-toxic, and neither biologically nor otherwise undesirable. For example, the additional components may be generally sterile and pyrogen free. Such components must be “acceptable” in the sense of being compatible with the composition of the invention and not deleterious to the recipients thereof. Thus, “pharmaceutically acceptable excipients” includes any compound(s) used in forming a part of the formulation that is intended to act merely as an excipient, i.e. not intended to have biological activity itself.

The nutraceutical, pharmaceutical, veterinary, oenological or cosmetic formulation/product may be in the form of a liquid or a solid.

Liquid dosage formulations/products for oral administration include solutions, emulsions, aqueous or oily suspensions, syrups and elixirs.

Formulations and products (e.g. pharmaceutical, veterinary or cosmetic formulations/products) described herein, such as those intended for oral administration, may be prepared according to methods known to those skilled in the art, such as by mixing the components of the formulation/product together.

The formulation or product (e.g. pharmaceutical, veterinary or cosmetic formulation/product) may contain one or more additional ingredients, such as pharmaceutical ingredients and excipients, such as sweetening agents, flavouring agents, colouring agents and preserving agents.

The formulation or product (e.g. pharmaceutical, veterinary or cosmetic formulation/product) may also contain one or more additional active ingredients, such as cosmetic or pharmaceutical active ingredients, such as hyaluronic acid, centella asiatica extract, peptides such as Matrixyl® and Argireline®, and mixtures thereof.

The formulation or product of the invention may contain the active ingredient(s) in admixture with non-toxic pharmaceutically acceptable excipients (or ingredients). These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof.

Liquid formulations or products (e.g. pharmaceutical, veterinary or cosmetic formulations/products) may be contained within a capsule, which may be uncoated or coated as defined above.

Suitable pharmaceutical or veterinary carriers include inert solid diluents or fillers, sterile aqueous solutions and various organic solvents. Examples of liquid carriers are syrup, peanut oil, olive oil, phospholipids, fatty acids, fatty acid amines, polyoxyethylene and water.

Moreover, the carrier or diluent may include any sustained release material known in the art, such as glyceryl monostearate or glyceryl distearate, alone or mixed with a wax.

Suitable pharmaceutical carriers include inert sterile aqueous solutions and various organic solvents. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Suitable cosmetic carriers are typically those that are suitable for topical administration to the outer surface of the human body, such as the skin and/or hair and/or scalp.

Typically, such carriers are dermatologically acceptable.

The phrase “dermatologically acceptable carrier” means that the carrier is suitable for topical application to the keratinous tissue, has good aesthetic properties, is compatible with the actives in the composition, and will not cause any unreasonable safety or toxicity concerns.

The carrier can be in a wide variety of forms. In some instances, the solubility or dispersibility of the components (e.g. extracts, sunscreen active, additional components) may dictate the form and character of the carrier. Non-limiting examples include simple solutions (e.g. aqueous or anhydrous), dispersions, emulsions, and solid forms (e.g. gels, sticks, flowable solids, or amorphous materials).

The dermatologically acceptable carrier may be in the form of an emulsion. An emulsion may be generally classified as having a continuous aqueous phase (e.g. oil-in-water and water-in-oil-in-water) or a continuous oil phase (e.g. water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g. water-soluble moisturizing agents, conditioning agents, anti-microbials, humectants and/or other skin care actives). However, in some instances, the aqueous phase may comprise components other than water, including but not limited to water-soluble moisturizing agents, conditioning agents, antimicrobials, humectants and/or other water-soluble skin care actives. In some instances, the non-water component of the composition comprises a humectant, such as glycerin and/or other polyol(s). Emulsions may also contain an emulsifier. Emulsifiers may be non-ionic, anionic or cationic.

The carrier may contain one or more dermatologically acceptable, hydrophilic diluents. As used herein, “diluent” includes materials in which the composition of the invention can be dispersed, dissolved, or otherwise incorporated. Hydrophilic diluents include water, organic hydrophilic diluents, such as lower monovalent alcohols (e.g., C₁-C₄), and low molecular weight glycols and polyols, including propylene glycol, polyethylene glycol, polypropylene glycol, glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

The cosmetic formulation/product may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the anti-glycation benefits provided by the composition.

In some instances, it may be desirable to select skin tone agents that function via different biological pathways so that the actives do not interfere with one another, which could reduce the efficacy of both agents. The additional ingredients, when incorporated into the composition, should be suitable for use in contact with human skin tissue without undue toxicity, incompatibility, instability, allergic response, and the like.

The term “carrier” as used herein, may also refer to a natural product or a product originating from nature that has been transformed or modified so that it is distinct from the natural product from which it originated, such as maltodextrin.

The amount of the composition of the invention present in nutraceutical, pharmaceutical, veterinary, oenological or cosmetic formulations or products will vary depending on the application.

Typically, the amount of composition of the invention that may be present in nutraceutical, pharmaceutical, veterinary, oenological or cosmetic formulations or products will be from about 0.001 to about 50% by weight, such as from about 0.01% to about 30% or from about 1% to about 20% of the nutraceutical, pharmaceutical, veterinary, oenological or cosmetic formulations or products, such as from about 0.01 to about 20%, or from about 0.1 to 10% or from about 1 to about 5% by weight of the formulation or product.

### Methods and Uses

As explained previously, the present inventors have surprisingly and unexpectedly found that combining carnosic acid with retinol reduces or prevents the oxidation of retinol and/or enhances the oxidative stability of retinol, in particular also during the formation of bio-retinol.

Thus, the present invention provides a method for reducing or preventing oxidation of retinol and/or enhancing the oxidative stability of retinol, wherein the method comprises combining retinol with carnosic acid.

The present invention also provides the use of carnosic acid in reducing or preventing oxidation of retinol and/or enhancing the oxidative stability of retinol.

In particular, the present invention provides a method of reducing or preventing oxidation of retinol and/or enhancing the oxidative stability of retinol, wherein carnosic acid is added to a culture medium comprising a microorganism capable of providing retinol.

As with the composition of the invention, the carnosic acid may be provided in the form of an extract obtained or obtainable from at least one plant selected from the Lamiaceae family, such as rosemary and/or sage as defined above.

In the method or use described herein, once combined the carnosic acid may be present in an amount of at least about 0.5% by weight of the combination.

In the method or use defined herein, the retinol and carnosic acid (for example, in the form of an extract obtained or obtainable from a plant of the Lamiaceae family as previously defined) may be combined in a cosmetic formulation, such as an anti-aging formulation.

As used herein, the term “reducing or preventing oxidation of retinol” is intended to mean that less than about 10% of the retinol present in the composition is oxidised to retinaldehyde and/or retinoic acid over a time period of at least 1 month, or at least 3 months or at least 6 months. For example, less than about 5% of the retinol present in the composition is oxidised to retinaldehyde and/or retinoic acid or less than 1% or less than 0.5% of the retinol present in the composition is oxidised to retinaldehyde and/or retinoic acid over a time period of at least 1 month, or at least 3 months or at least 6 months.

As used herein, the term “enhances the oxidative stability of retinol” is intended to mean that at least 90% of the retinol present in the composition is still in the form of retinol over a time period of at least 1 month, or at least 3 months or at least 6 months. For example, more than 95% of the retinol present in the composition is still in the form of retinol or more than 99% or 99.5% of the retinol present in the composition is still in the form of retinol over a time period of at least 1 month, or at least 3 months or at least 6 months.

The present invention will be further described with reference to the following, non-limiting examples:

### EXAMPLE 1: PREPARATION OF A COMPOSITION OF THE INVENTION

72.9 kg of sunflower oil was heated at 50-55° C. and 8.8 kg of a hydro-ethanolic rosemary extract added. The mixture was stirred until complete dissolution of the rosemary extract. The mixture was then subjected to hot filtration on a Fibrafix® filter plate AF6 (retention rate 35-15 μm) with 1% of perlite. The carnosic acid content of the mixture was then quantified and adjusted with additional oil if required in order to obtain 4% carnosic acid by weight of the mixture.

45 kg of retinol and 55 kg of the oil mixture obtained above were then mixed at 35° C.

### EXAMPLE 2: OXIDATIVE STABILITY OF RETINOL IN THE COMPOSITIONS OF THE INVENTION COMPARED TO A RETINOL COMPOSITION COMPRISING BHT

Retinol oxidises irreversibly to retinoic acid. In order to assess the oxidative stability of the compositions of the invention, compositions within the scope of the invention and compositions comprising BHT only were tested at 1, 3 and 6 months to ascertain if any retinol oxidation had occurred.

As shown in the Tables 1 to 4 below, the compositions comprising retinol and carnosic acid or retinol, carnosic acid and BHT were able to stabilize retinol at a similar level to retinol composition that comprised BHT alone as the antioxidant. These stability tests were run at room temperature (RT), lower temperature (4° C.) and higher temperature (40° C.), respectively.

TABLE 1
Stability of Composition Comprising Retinol, Sunflower oil, and BHT

| | | Test | | | | |
|---|---|---|---|---|---|---|
| Component | Unit | method | T0 | T + 1 month | T + 3 months | T + 6 months |
| Retinol - RT | wt % | HPLC | 45.51 | 34.19 | 41.13 | 38.82 |
| Retinol - 4° C. | wt % | HPLC | | 35.06 | 41.78 | 41.65 |
| Retinol - 40° C. | wt % | HPLC | | 31.97 | 31.89 | 21.44 |
| all-Trans retinol - RT | Area % | HPLC | 96.53 | 96.23 | 95.47 | 95.24 |
| all-Trans retinol - 4° C. | Area % | HPLC | | 98.66 | 96.15 | 95.90 |
| all-Trans retinol - 40° C. | Area % | HPLC | | 94.8 | 92.08 | 90.19 |
| 13-cis retinol - RT | Area % | HPLC | 3.47 | 3.77 | 4.53 | 4.76 |
| 13-cis retinol - 4° C. | Area % | HPLC | | 1.34 | 3.85 | 4.10 |
| 13-cis retinol - 40° C. | Area % | HPLC | | 5.2 | 7.92 | 9.81 |
| Retinoic acid - RT | ppm | HPLC | Not detected | Not detected | Not detected | Not detected |
| Retinoic acid - 4° C. | ppm | HPLC | | Not detected | Not detected | Not detected |
| Retinoic acid - 40° C. | ppm | HPLC | | Not detected | Not detected | Not detected |
| Retinal - RT | wt % | HPLC | 0.23 | 0.14 | Not detected | Not detected |
| Retinal - 4° C. | wt % | HPLC | | 0.13 | Not detected | Not detected |
| Retinal - 40° C. | wt % | HPLC | | Not detected | Not detected | Not detected |
| BHT - RT | wt % | GC/FID | 4.2 | Not determined | Not determined | 4.2 |
| BHT - 4° C. | wt % | GC/FID | | | | |
| BHT - 40° C. | wt % | GC/FID | | | | |

TABLE 2
Stability of Composition Comprising Retinol, Sunflower
oil, Rosemary Extract (2.25%) and BHT (2.25%)

| | | Test | | | | |
|---|---|---|---|---|---|---|
| Component | Unit | method | T0 | T + 1 month | T + 3 months | T + 6 months |
| Retinol - RT | wt % | HPLC | 45.40 | 34.11 | 39.35 | 37.15 |
| Retinol - 4° C. | wt % | HPLC | | 35.64 | 43.35 | 42.37 |
| Retinol - 40° C. | wt % | HPLC | | 28.51 | 24.45 | 15.01 |
| all-Trans retinol - RT | Area % | HPLC | 99.35 | 96.3 | 95.9 | 95.89 |
| all-Trans retinol - 4° C. | Area % | HPLC | | 96.4 | 96.2 | 96.21 |
| all-Trans retinol - 40° C. | Area % | HPLC | | 98.5 | 96.6 | 91.77 |
| 13-cis retinol - RT | Area % | HPLC | 0.65 | 3.7 | 4.1 | 4.11 |
| 13-cis retinol - 4° C. | Area % | HPLC | | 3.6 | 3.8 | 3.79 |
| 13-cis retinol - 40° C. | Area % | HPLC | | 1.5 | 3.4 | 8.23 |
| Retinoic acid - RT | ppm | HPLC | Not detected | Not detected | Not detected | Not detected |
| Retinoic acid - 4° C. | ppm | HPLC | | Not detected | Not detected | Not detected |
| Retinoic acid - 40° C. | ppm | HPLC | | Not detected | Not detected | Not detected |
| Retinal - RT | wt % | HPLC | 0.25 | 0.10 | Not detected | Not detected |
| Retinal - 4° C. | wt % | HPLC | | 0.14 | Not detected | Not detected |
| Retinal - 40° C. | wt % | HPLC | | 0.06 | Not detected | Not detected |
| BHT - RT | wt % | GC/FID | 2.1 | Not determined | Not determined | Not determined |
| Carnosic acid - RT | wt % | HPLC | 1.1 | Not determined | Not determined | Not determined |
| | | (1961) | | | | |

TABLE 3
Stability of Composition Comprising Retinol, Sunflower Oil, and Rosemary Extract (9.8%)

| | | Test | | | | |
|---|---|---|---|---|---|---|
| Component | Unit | method | T0 | T + 1 month | T + 3 months | T + 6 months |
| Retinol - RT | wt % | HPLC | 48.36 | 40.52 | 37.19 | 35.46* |
| Retinol - 4° C. | wt % | HPLC | | 45.56 | 40.52 | 42.70 |
| Retinol - 40° C. | wt % | HPLC | | 22.47 | 7.00 | 0.84 |
| all-Trans retinol - RT | Area % | HPLC | 99.5 | 99.3 | 99.3 | 99.1 |
| all-Trans retinol - 4° C. | Area % | HPLC | | 95.0 | 99.5 | 99.5 |
| all-Trans retinol - 40° C. | Area % | HPLC | | 96.9 | 94.8 | 85.4 |
| 13-cis retinol - RT | Area % | HPLC | 0.5 | 0.7 | 0.7 | 0.9 |
| 13-cis retinol - 4° C. | Area % | HPLC | | 5.0 | 0.5 | 0.5 |
| 13-cis retinol - 40° C. | Area % | HPLC | | 3.1 | 5.2 | 14.6 |
| Retinoic acid - RT | ppm | HPLC | Not detected | Not detected | Not detected | Not detected |
| Retinoic acid - 4° C. | ppm | HPLC | | Not detected | Not detected | Not detected |
| Retinoic acid - 40° C. | ppm | HPLC | | Not detected | Not detected | Not detected |
| Retinal - RT | wt % | HPLC | Not detected | Not detected | Not detected | Not detected |
| Retinal - 4° C. | wt % | HPLC | | Not detected | Not detected | Not detected |
| Retinal - 40° C. | wt % | HPLC | | Not detected | Not detected | Not detected |
| Carnosic acid - RT | wt % | HPLC | 4.6 | Not determined | Not determined | Not determined |
| | | (1961) | | | | |

TABLE 4
Stability of Composition Comprising Retinol, Sunflower Oil, and Rosemary Extract (4.2%)

| | | Test | | | | |
|---|---|---|---|---|---|---|
| Component | Unit | method | T0 | T + 1 month | T + 3 months | T + 6 months |
| Retinol - RT | wt % | HPLC | 47.21 | 44.35 | 41.24 | 42.53 |
| Retinol - 4° C. | wt % | HPLC | | 46.29 | 40.95 | 52.97 |
| Retinol - 40° C. | wt % | HPLC | | 34.66 | 21.92 | 9.01 |
| all-Trans retinol - RT | Area % | HPLC | 99.6 | 94.8 | 99.4 | 99.3 |
| all-Trans retinol - 4° C. | Area % | HPLC | | 95.4 | 99.5 | 99.5 |
| all-Trans retinol - 40° C. | Area % | HPLC | | 98.5 | 97.4 | 94.6 |
| 13-cis retinol - RT | Area % | HPLC | 0.4 | 5.2 | 0.6 | 0.7 |
| 13-cis retinol - 4° C. | Area % | HPLC | | 4.6 | 0.5 | 0.5 |
| 13-cis retinol - 40° C. | Area % | HPLC | | 1.5 | 2.6 | 5.4 |
| Retinoic acid - RT | ppm | HPLC | Not detected | Not detected | Not detected | Not detected |
| Retinoic acid - 4° C. | ppm | HPLC | | Not detected | Not detected | Not detected |
| Retinoic acid - 40° C. | ppm | HPLC | | Not detected | Not detected | Not detected |
| Retinal - RT | wt % | HPLC | Not detected | Not detected | Not detected | Not detected |
| Retinal - 4° C. | wt % | HPLC | | Not detected | Not detected | Not detected |
| Retinal - 40° C. | wt % | HPLC | | Not detected | Not detected | Not detected |
| Carnosic acid - RT | wt % | HPLC | 1.91 | Not determined | Not determined | Not determined |
| | | (1961) | | | | |

1. A process of preparing a composition comprising retinol, an oil and carnosic acid, the process comprising the step of culturing a microorganism capable of providing retinol in a culture medium comprising an oil so that retinol accumulates in the oil, wherein carnosic add is added to the culture medium.

2. The process of claim 1, wherein the carnosic acid is added to the culture medium at the start of and/or during the culturing.

3. The process of claim 1, wherein the carnosic acid is added in an amount that is not toxic to the microorganism.

4. The process of claim 1, further comprising the step of isolating the composition comprising the retinol, oil and carnosic acid from the cultured microorganism and/or the culture medium.

5. The process of claim 1, wherein the carnosic acid is in the form of an extract obtained or obtainable from at least one plant selected from the Lamiaceae family.

6. The process of claim 5, wherein the carnosic content in the extract obtained or obtainable from rosemary is at least about 20% by weight of the rosemary extract.

7. The process of claim 1, wherein the microorganism is cultured in a culture medium comprising one or more plant oils or vegetable oils selected from one or more of coconut oil, palm oil, cottonseed oil, wheat germ oil, soybean oil, sesame oil, olive oil, corn oil, sunflower oil, safflower oil, peanut oil, flaxseed oil, grape seed oil, rapeseed oil, and mixtures thereof.

8. The process of claim 1, wherein the microorganism is selected from the group consisting of Yarrowia lipolytica and Saccharomyces cerevisiae, which may optionally be genetically modified to optimize the production of retinol.

9. The process of claim 1, wherein the microorganism is cultured in a bioreactor containing an aqueous phase and an oily extraction phase.

10. The process of claim 1, wherein the composition comprising the retinol, oil and carnosic acid is a cosmetic composition.

11. The process of claim 1, further comprising the step of formulating the composition comprising the retinol, oil and carnosic acid into a formulation.

12. A cosmetic composition formed by the process of claim 1.

13. A method of reducing or preventing oxidation of retinol and/or enhancing the oxidative stability of retinol, wherein carnosic acid is added to a culture medium comprising a microorganism capable of providing retinol.

14. The process of claim 5, wherein the carnosic acid is in the form of an extract obtained or obtainable from rosemary.

15. The process of claim 14, wherein the carnosic acid is in the form of a rosemary leaf extract.

16. The process of claim 14, wherein the extract is a hydro-ethanolic extract.

17. The process of claim 14, wherein the extract is a hydro-ethanolic rosemary leaf extract.

18. The process of claim 6, wherein the carnosic content in the extract obtained or obtainable from rosemary is at least about 30% by weight of the rosemary extract.

19. The process of claim 11, wherein the formulation is a cosmetic formulation.

20. The process of claim 19, wherein the formulation is an anti-aging formulation.
